(19) United States
(12) Patent Application Publication
(10) Pub. No.: US 2012/0064521 A1
(43) Pub. Date: Mar. 15, 2012

(54) DETECTION OF DNA HYDROXYMETHYLATION

(76) Inventors: James Yen, Garden Grove, CA (US); Xiyu Jia, Newport Beach, CA (US)

(21) Appl. No.: 12/963,272

(22) Filed: Dec. 8, 2010

Related U.S. Application Data
(60) Provisional application No. 61/381,228, filed on Sep. 9, 2010.
(60) Provisional application No. 61/392,932, filed on Oct. 13, 2010.

(51) Int. Cl. C12Q 1/68; C07H 21/04

(52) U.S. Cl. 435/6.11; 536/23.1

(57) ABSTRACT

Reagents and methods for analysis of DNA hydroxymethylation are provided. Methods comprise modification of hydroxymethylated cytosine residues with a bulky moiety to protect hydroxymethylated positions from cleavage with a DNA endonuclease. For example, methods may comprise contacting DNA with a glucosyltransferase to glucosylate hydroxymethylated DNA positions and digesting the DNA with a DNA endonuclease to cleave DNA in positions lacking hydroxymethylation. Reagents and kits for hydroxymethylated DNA analysis are also provided.

This application claims the priority of U.S. Provisional Application No. 61/381,228, filed Sep. 9, 2010, and U.S. Provisional Application No. 61/392,932, filed Oct. 13, 2010, the entire disclosures of which are incorporated herein by reference.

### BACKGROUND OF THE INVENTION

1. Field of the Invention

The present invention generally relates to molecular biology. More specifically, the invention relates to methods and compositions for genomic DNA hydroxymethylation analysis.

2. Description of the Related Art

Epigenetic modifications are regarded as fundamental elements in gene expression regulation. DNA methylation, one such modification, plays crucial roles in widespread biological phenomena including host defense in bacteria and cell cycle regulation, gene imprinting, embryonic development and X-chromosome inactivation in mammals. Aberrant DNA methylation patterns in gene promoters are closely associated with perturbations in gene expression and have recently been indicated as leading cause of human cancers (Jones and Laird, 1999).

The field of epigenetics has grown exponentially in the scientific community as irregularities with gene expression due to abnormal DNA methylation is the leading cause in human cancer types. DNA methylation involves the chemical addition of a methyl group to the 5′ carbon position on the cytosine pyrimidine ring. Most DNA methylation occurs within CpG islands which are commonly found in the promoter region of a gene. Thus, this form of post modification of DNA acts as communicative signal for activation or inactivation of certain gene expression throughout various cell types.

The existence of 5′-hydroxymethylcytosine (5′hmC) was classically only known to exist in T-even bacteriophages (T2/T4/T6). Recently, this ultra-modified base was identified in mammalian tissue (i.e., brain and embryonic stem cells). Until now, only global quantification of this base was possible, using such techniques such as HPLC, thin layer chromatography (TLC), and LC/MS. Site specific detection or sequence context detection of 5′hmC has been a challenge because existing techniques to study 5′-methylcytosine (5′mC) in a site specific manner (bisulfite conversion) cannot distinguish between 5′mC and 5′hmC.

### SUMMARY

In a first embodiment there is provided a method detecting DNA hydroxymethylation in a DNA sample comprising (i) obtaining a DNA sample comprising at least a first 5′hmC position that has been modified by the addition of a bulky chemical moiety; and (ii) contacting the DNA sample with a DNA endonuclease (e.g., a methylation dependent DNA endonuclease) to cleave the DNA, wherein the bulky chemical moiety blocks cleavage of the DNA at 5′hmC position(s). Cleaved DNA samples can then be analyzed to detect at least a first DNA sequence from the sample that is not cleaved by the DNA endonuclease to determine the presence of hydroxymethylation in the DNA sequence. In certain aspects, the DNA sample can be contacted with two, three, four, or more DNA endonucleases. Uncleaved DNA positions comprising a modified 5′hmC can be detected by any of an array of DNA analysis techniques that are known in the art including, but not limited to, DNA sequencing and hybridization (e.g., hybridization to an oligonucleotide array). Thus, in certain aspects, methods according to the invention can be used to determine the presence of DNA hydroxymethylation at a plurality of potential hydroxymethylation sites, such as at least 5, 10, 15, 20, 50, 100, 500 or 1,000 potential hydroxymethylation sites in a DNA sample. In a further aspect, determining the presence of DNA hydroxymethylation at a potential methylation site comprises identifying a sequence corresponding to a detected DNA sequence on a genomic map to, for example, identify hydroxymethylation in gene expression control regions (e.g., promoters, enhancers, or splice regulator sequences) or in protein coding sequences. DNA endonucleases for use according to the invention include, but are not limited to MspI, BisI, GlaI, Csp6I, HaeIII, TaqI (e.g., TaqαI), MboI, Hpy188I, HpyCH4III or McrBC.

In a further embodiment there is provided a method detecting DNA methylation and hydroxymethylation in a DNA sample comprising at least a first 5′hmC that has been modified by the addition of a bulky chemical moiety. Such a method comprises (i) contacting a DNA sample with a methylation sensitive DNA endonuclease (MSE) to cleave DNA at positions lacking a 5′mC or 5′hmC; (ii) contacting the cleaved DNA sample with a methylation dependent DNA endonuclease to further cleave the DNA at positions comprising a 5′mC; and (iii) detecting DNA sequences not cleaved by the methylation dependent DNA endonuclease and the MSE to determine the presence of hydroxymethylation and methylation in the sample. For example, analysis after cleavage with a MSE can be used to determine positions that are methylated or hydroxymethylated, whereas positions not cleaved by the methylation dependent DNA endonuclease are indicative specifically of positions that are hydroxymethylated. Thus, analysis of the DNA sequences not cleaved at these two steps can be used to determine which DNA positions comprise 5′mC and which comprise 5′hmC.

In certain embodiments, DNA samples for use according to the invention are subjected to additional treatment prior to determining the presence of 5′mC or 5′hmC at positions in the sample. For example, DNA may be substantially purified to remove contaminants that may interfere with downstream enzymatic or chemical process such a DNA cleavage or PCR. In some cases, DNA may be sheared to reduce the size of DNA molecules and/or the viscosity of a sample. For example, DNA samples can be sheared by mechanical shearing, sonication or treatment with endonuclease. In still further aspects, a DNA sample may be treated to methylate cytosine positions prior to cleaving thereby rendering additional sites cleavable by a methylation dependent DNA endonuclease. For instance, a DNA sample may be treated with a methyl transferase such as a M.SssI and/or M.CviPI methyltransferase.

In some aspects, methods of the invention concern contacting a DNA sample with a methylation dependent DNA endonuclease under conditions (e.g., proper salt, buffer, and temperature conditions) wherein the endonuclease cleaves DNA at recognition sites comprising a 5′mC, but not at sites comprising a modified 5′hmC. In some cases, two or more methylation dependent DNA endonuclease enzymes are used that comprise different recognition sites. For example, the methylation dependent DNA endonuclease can be BisI, GlaI or McrBC or a mixture thereof.

In certain embodiments, DNA samples for use according to the invention comprise at least a first 5′hmC position that has been modified by the addition of a bulky chemical moiety. Examples of bulky chemical moieties include, but are not limited to, hydrocarbon chains, aromatic rings, saturated and unsaturated lipids, sugars, polysaccharides and amino acids. For instance, 5′hmC positions may be glycosylated, such as by additional of a glucose moiety (i.e., glucosylated). Thus, according to certain aspects of the invention, 5′hmC positions in sample DNA are modified by a chemical or enzymatic process. In some aspects, a DNA sample is treated with an enzyme to glycosylate 5′hmC. For example, a DNA sample can be treated to glucosylate hydroxymethylcytosine positions such as by contacting the DNA with a glucosyltransferase. A glucosyltransferase can be produced recombinantly or may be directly purified (e.g., from a bacterial cell infected with a T-even bacteriophage). For example, a glucosyltransferase may be an a-glucosyltransferase or a β-glucosyltransferase, such as a β-glucosyltransferase from a T4 bacteriophage encoded by a nucleic acid according to SEQ ID NO: 3.

In certain embodiments, methods for determining the presence of hydroxymethylation involve ligating cleaved DNA to one or more oligonucleotide tags to generate tagged DNA(s). In certain aspects, oligonucleotide tag sequences comprise double stranded DNA having a known sequence, such a sequence that hybridizes to primers that can be used for DNA sequencing and/or PCR amplification. For example, methods for DNA methylation analysis by tagging cleaved DNA are known in the art and may be applied to methods according to the invention (see, e.g., WO/2010/114821, incorporated herein by reference). In some aspects, oligonucleotide tag sequences comprise a label, such as a fluorescent label, a colorimetric label, a radioactive label, an antigen label, a sequence label, an enzymatic label or an affinity label (e.g., biotin). Thus, in certain cases, tagged DNA can be purified using the label, such as by using an avidin-biotin affinity column or affinity beads. A variety of commercially available ligase enzymes may be employed for ligating cleaved DNA to tags, including but not limited to, a bacterial DNA ligase or a phage DNA ligase (e.g., T4 DNA ligase). In further aspects, methods according to the invention further comprise treating the ligated (tagged) DNA with an enzyme that polymerizes additional 3′ sequence, thereby repairing the 3′ end of the tagged DNA. For example, a DNA polymerase such as Taq polymerase can be employed.

DNA samples for use according the invention can be from any source that potentially comprises DNA with hydroxymethylated cytosines. For example, a DNA sample can comprise mammalian genomic DNA, such as human genomic DNA. DNA may be from, for example, a human subject, a tissue culture cell or cell line or a tissue bank. A DNA sample from a patient or subject may be isolated from, for example, a blood sample, a tissue biopsy sample, a urine sample a saliva sample, or a skin sample. In some aspects, methods according to the invention may involve comparing hydroxymethylation status in two or more DNA samples to determine differential DNA hydroxymethylation between two or more samples. For example, a sample from a tumor may be compared to a sample from surrounding tissue or samples collected over a period of time may be compared to determine changes in hydroxymethylation status over time. In still a further example, samples for comparison can be from tissue culture cells grown under different conditions; from cells at different stages of differentiation; from healthy and diseases tissue; samples two or more different organisms or individuals; or from cells treated with a drug and placebo. In certain aspects two samples that are analyzed may be a test sample and a control sample. For example, a control sample may be DNA that does not comprise a bulky moiety (e.g., glucose) that blocks 5′hmC positions. In still further aspects, a control DNA sample may comprise a known level of DNA methylation or DNA hydroxymethylation, such as DNA from a cell line that lacks methyltransferase enzymes (unmethylated DNA), or DNA that has been treated to methylate or hydroxymethylate essentially all positions in the sample.

In yet a further embodiment, the invention provides a method for enriching hydroxymethylated DNA in a sample comprising (i) contacting the DNA sample with a glucosyltransferase to glucosylate hydroxymethylcytosines; and (ii) contacting the glucosylated DNA sample with one or more DNA endonuclease (e.g., one or more methylation dependent DNA endonucleases) to cleave the DNA. In certain aspects, a DNA sample is first treated with a methyltransferase enzyme to methylate additional cytosine positions, thereby further enriching the sample for sequence comprising hydroxymethylated cytosines.

In still a further embodiment, the invention provides kits for analysis of DNA hydroxymethylation. In one aspect a kit may comprise reagents for analysis of total DNA hydroxymethylation levels by labeling 5′hmC positions with a labeled glucose (such as labeled uridine diphosphate glucose (UDPG)). Such kits comprise an active glucosyltransferase, such as β-glucosyltransferase, and a labeled glucose enzyme substrate. In a further aspect, kits are provided for determining one or more hydroxymethylated positions in a DNA sample. For example, a kit can comprise, at least, an active glucosyltransferase and a DNA endonuclease (e.g., MspI, TaqI or a methylation dependent DNA endonuclease, such as BisI, GlaI or McrBC). Kits according to the invention can further comprise one or more MSEs; a DNA methyltransferase (e.g., M.SssI and/or M.CviPI methyltransferase); an enzyme that converts 5′mC into 5′hmC (e.g., recombinant Tet1, Tet2 and/o Tet3 proteins); one or more reference DNA samples; an affinity purification column; a DNA ligase; a DNA polymerase; DNA sequencing reagents; a glucosylation buffer; UDPG; a PCR buffer; instructions; methylation or hydroxymethylation specific antibodies; and/or DNA primers.

In yet still a further embodiment, the invention provides an antibody or fragment thereof that binds to a 5′-glucosylated hydroxymethylcytosine. For example, a 5′-glucosylated hydroxymethylcytosine-binding antibody can be a polyclonal or monoclonal antibody, such as a full-length antibody, chimeric antibody, Fab′, Fab, F(ab′)2, single domain antibody (DAB), Fv, or a single chain Fv (scFv). In a certain aspects, a 5′-glucosylated hydroxymethylcytosine-binding antibody can be used in a method for determining the presence glucosylated hydroxymethylcytosine (i.e., corresponding to a hydroxymethylated DNA position) in a DNA sample comprising: (i) contacting the DNA sample with the antibody; and (ii) detecting antibody binding to determine the presence of glucosylated hydroxymethylcytosine in the sample. Further methods for making and using such antibodies are detailed below.

As used herein, “a” or “an” may mean one or more. As used herein in the claim(s), when used in conjunction with the word “comprising”, the words “a” or “an” may mean one or more than one.

The use of the term “or” in the claims means “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWING

The following drawings are part of the present specification and are included to further demonstrate certain aspects of the present invention. The invention may be better understood by reference to the drawings in combination with the detailed description of specific embodiments presented herein.

FIG. 1: Methylation dependent enzymes recognize both 5′mC and 5′hmC modified DNA. PCR products having the same primary sequence and differing only in the modification status of cytosines were digested with the indicated endonucleases and analyzed by agarose gel electrophoresis. The methylation status of all cytosines in the analyzed DNA molecules are indicated as unmodified (C), 5′-methylcyotine (mC), or are 5-hydroxymethylcytosine (^{hm}C). Digestions were carried out for 3 hours at recommended enzyme reaction conditions.

FIG. 2A-B: Transfer of a glucose group via β-glucosyltransferase blocks endonuclease digestion of DNA and is specific for 5′hmC. PCR products having the same primary sequence and differing only in the modification status of cytosines were digested as indicated with MspI (FIG. 2A) or GlaI (FIG. 2B) and analyzed by agarose gel electrophoresis. The methylation status of all cytosines in the analyzed DNA molecules are indicated as unmodified (C), 5′-methylcyotine (^{m}C), or are 5-hydroxymethylcytosine (^{hm}C). Digestions carried out for 3 hours at recommended enzyme reaction conditions. +Beta-GT denotes DNA in vitro glucosylated with T4 β-glucosyltransferase.

FIG. 3: DNA template with hemi-Glu-hmC effectively blocks MspI digestion. DNA molecules including a hemi-hydroxymethylcytosine motif within a MspI recognition site CCGG at the internal C (indicated by underlining) were digested as indicated and analyzed by agarose gel electrophoresis. “Untreated template” is the hemi-hydroxymethylcytosine DNA template undigested. “+Glucosylation” is a DNA template in vitro glucosylated with β-glucosyltransferase. “−Control” is mock glucosylated in a reaction treated without a β-glucosyltransferase enzyme.

FIG. 4: DNA templates comprising Glu-hmC display hindered TakαI digestion. DNA templates containing 100% of the cytosines modified to 5′-hydroxymethylcytosine (hmC) were glucosylated in vitro by β-glucosyltransferase (Glu-hmC). DNA templates comprising each modification were digested with TaqI following recommended conditions and samples were taken at indicated time points (time indicated in minutes) and analyzed by agarose gel electrophoresis.

FIG. 5: Glu-hmC blocks MspI digestion in CpG context. A DNA template containing all unmodified cytosines (untreated sample) was in vitro methylated at CpG sites with M.SssI (control (mC)). CpG methylated template was treated in vitro with Tet1 to create 5′-hydroxymethylcytosine on the premethylated (5′mCpG) sites. Then mC and mC+Tet1 (5′hmC) samples were glucosylated with β-glucosyltransferase and subsequently digested with MspI. Only the DNA treated with Tet1 contains 5′hmC which could accept a glucose moiety. The different cutting patterns (protection from digestion) of MspI indicates the presence of Glu-5′hmC.

FIG. 6: DNA comprising glucosyl-5′-hydroxymethylcytsoine can be amplified by PCR. DNA was amplified from pUC18 using primers pUC 5′ (SEQ ID NO: 4) and pUC 3′ (SEQ ID NO: 5). Amplified PCR product was left untreated “C”; in vitro methylated with M.SssI “mC”; or in vitro methylated, hydroxymethylated with Tet1 and glucosylated with β-glucosyltransferase “GluhmC”. qRT-PCR was performed on the sample in duplicate. The resulting amplification curves are shown in graphical format.

FIG. 7: 5′-hmC glucosyltransferase transfers a glucose moiety from uridine diphosphoglucose (UDPG) onto preexisting 5′-hydroxymethylcytosines within DNA.

FIG. 8: Treatment of DNA containing 5′hmC with 5′-hmC glucosyltransferase specifically adds a glucose moiety yielding glucosyl-5′-hydroxymethylcytosine. Subsequent digestion with glucosyl-5-hydroxymethylcytosine sensitive endonucleases will cut DNA with 5-methylcytosine or 5′-hydroxymethylcytosine in their recognition sequence, but leave glucosyl-5-hydroxymethylcytosine DNA uncleaved.

### DETAILED DESCRIPTION OF THE INVENTION

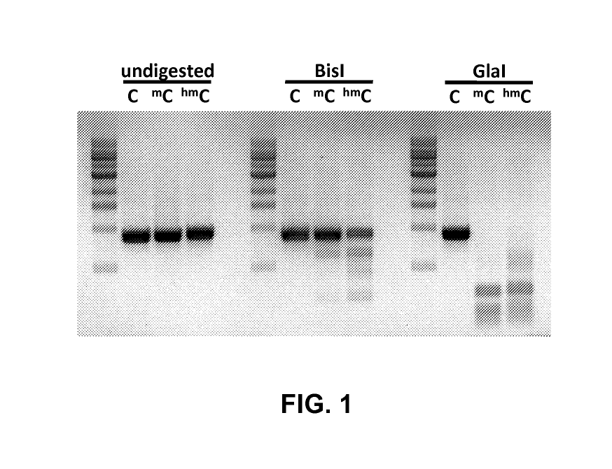

Genomic DNA methylation and hydroxymethylation are emerging as key epigenetic regulators of gene expression, especially in higher organisms such as humans. However, analysis of these modifications and their role in gene regulation has been hampered the inability of standard DNA analysis techniques to distinguish between DNA positions comprising these modifications. In particular, available techniques for analysis of methylated DNA, such as bisulfate sequencing and use of methylation sensitive endonucleases, are unable to distinguish between methylated and hydroxymethylated cytosines (see, e.g., Nestor et al., 2010 and Huang et al., 2010; FIG. 1). To date the only techniques for examining DNA hydroxymethylation, such as thin layer chromatography and the use of 5′hmC binding antibodies, have proven inadequate for sequence specific analysis of DNA.

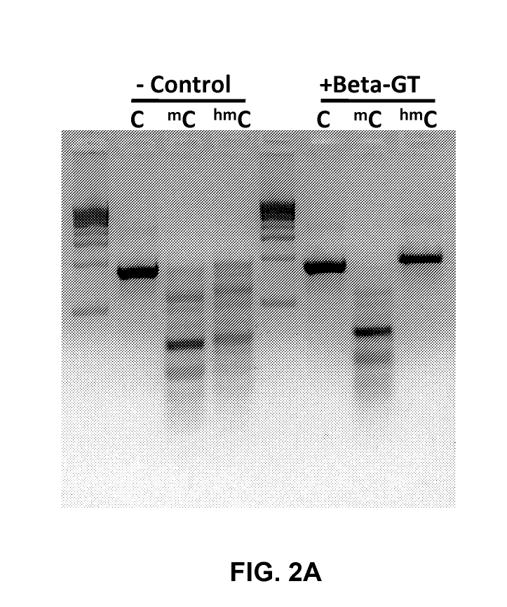

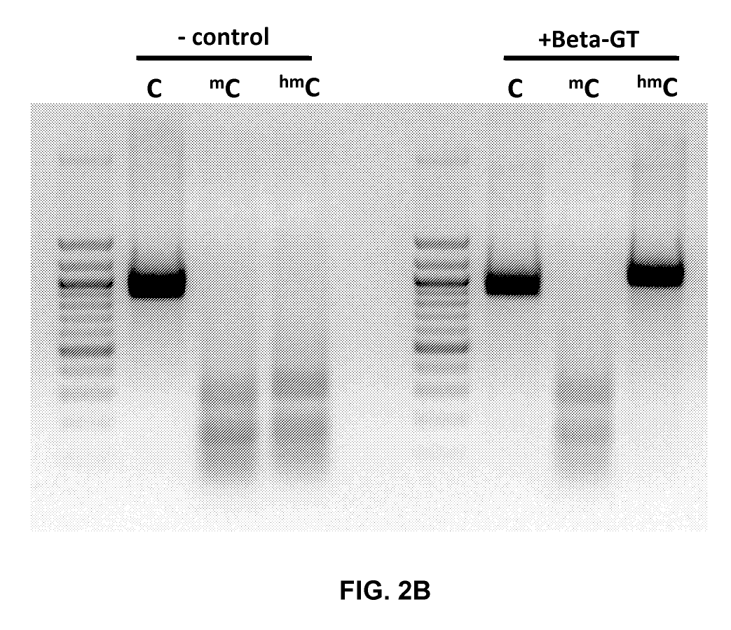

Techniques and regents detailed in the instant application allow efficient analysis of epigenetic modification to DNA and are able to distinguish between methylated and hydroxymethylated cytosine position. For example, reagents detailed here are able to mediate highly efficient glucosylation of DNA at 5′hmC positions (FIG. 2). The glucosylation reaction was demonstrated to be specific for 5′hmC and nonspecific modification of cytosines lacking hydroxymethylation was not observed. The additional of the bulky sugar moiety at the 5′hmC was found to effectively inhibit cleavage of the DNA by DNA endonucleases including those that require methylation Inhibition of endonuclease activity was observed even in the case of hemi-glucsylated DNA molecules (where only one cytosine of one strand included the modification). Thus, protection of 5′hmC positions provides a method for specific analysis of hydroxymethylation versus cytosine positions that are methylation or lack modification.

A variety of methods can be used to analyze DNA molecules comprising a protected 5′Glu-hmC position. For example, glucosylated DNA can be hybridized to an array to determine the sequences of hydroxymethylated positions in DNA samples. DNA molecules comprising a 5′Glu-hmC were also found to serve a suitable template for DNA polymerase (See e.g., Example 6). Accordingly, protected DNA molecules can also by analyzed by PCR-based techniques or by direct DNA sequencing. Furthermore, modified 5′Glu-hmC may provide antibody-binding target and 5′Glu-hmC-binding antibodies may exhibit enhanced specificity and binding affinity relative to antibodies that bind to 5′hmC. Accordingly, 5′Glu-hmC-binding antibodies can be used in improved methods DNA hydroxymethylation analysis.

The following is a detailed description of the invention provided to aid those skilled in the art in practicing the present invention. Those of ordinary skill in the art may make modifications and variations in the embodiments described herein without departing from the spirit or scope of the present invention.

### I. GENERAL PROTOCOL

An illustrative and non-limiting protocol for hydroxymethylation analysis according to the invention is exemplified below.

1. Modifying hydroxymethylcytosine positions in a DNA sample. A DNA sample used for analysis may be chemically modified or treated with and enzyme to modify any hydroxymethylcytosine positions that are present in the sample. For example, efficient glucosylation of hydroxymethylcytosine can be achieved by incubating a sample DNA with a glucosyltransferase enzyme, such as a glucosyltransferase from a T2, T4 or T6 bacteriophage. In certain aspects a sample may be split and one portion of the sample treated with a glucosyltransferase (a test sample) while another portion is mock treated (a control sample).

Optionally, a DNA sample may be treated with a DNA methyltransferase prior to step 1, thereby methylating essentially all potential sites of methylation.

2. Contact the DNA sample with a DNA endonuclease. Once the hydroxymethylated DNA positions have been protected from enzyme cleavage by modification (e.g., by glucosylation). DNA is contact with one or more DNA endonuclease enzyme(s). The enzyme(s) cleave at their corresponding recognition sites if no blocking moiety is present, while recognition sites with a 5′hmC are protected from cleavage by their previous modification. For example, in the case of glucosylation of 5′hmC, an endonuclease for use according to the invention displays differential sensitivity when glucosyl-5-hydroxymethylcytosine is present within its recognition sequence, versus unmodified cytosine, 5-methylcytosine, or 5-hydroxymethylcytosine. An example is an endonuclease that will be able to cleave at sequences comprising an unmodified cytosine, 5-methylcytosine, or 5-hydroxymethylcytosine but cannot digest glucosyl-5-hydroxymethylcytosine. Some non-limiting examples of such DNA endonuclease enzymes include MspI, GlaI, Csp6I, HaeIII, TagαI, MboI, McrBC, Hpy188I and HpyCH4III.

3. Determine hydroxymethyalted DNA positions in the DNA sample. Cleaved DNA is analyzed, for example to identify sequences that were not cleaved but have a recognition site for a methylation dependent DNA endonuclease used in the cleavage reaction. The presence of an intact site is indicative a site that was hydroxymethylated in the DNA sample.

A wide range of analysis techniques may be used to determine hydroxymethylation in a sample. For example, methods for analysis include:
Ligating the cleaved DNA to an oligonucleotide tag comprising a detectable label and hybridizing the tagged DNA(s) to an array of known sequences to identify positions of hydroxymethylation.
Ligating the cleaved DNA oligonucleotide tags having known sequences. The tagged DNAs can then be amplified by PCR (e.g., for sequencing or cloning) or directly sequenced.
Hybridizing the cleaved DNA to one or more labeled probe wherein hybridization is indicative of positions with hydroxymethylation.
The hydroxymethylation status of a specific sequence of set of sequences need to be determined the cleaved DNA can subjected to PCR where amplification of a product comprising a potential site of hydroxymethylation is indicative of the presence of hydroxymethylation. In certain aspects quantitative PCR may be used to quantify the level or proportion of DNA in a sample that comprises hydroxymethylation at a given position.

### II. GENOMIC DNA AND SAMPLES

Exemplary DNA samples that can be used in a method of the invention include, without limitation, mammal DNA such as a rodent, mouse, rat, rabbit, guinea pig, ungulate, horse, sheep, pig, goat, cow, cat, dog, primate, human or non-human primate. Plant DNA may also be analyzed according to the invention. For example, DNA from Arabidopsis thaliana, maize, sorghum, oat, wheat, rice, canola, or soybean may be analyzed. It is further contemplated that genomic DNA from other organisms such as algae, a nematodes, insects (e.g., Drosophila melanogaster, mosquito, fruit fly, honey bee or spider), fish, reptiles, amphibians and yeast may be analyzed.

As indicated above, DNA such as genomic DNA can be isolated from one or more cells, bodily fluids or tissues. An array of methods can be used to isolate DNA from samples such as blood, sweat, tears, lymph, urine, saliva, semen, cerebrospinal fluid, feces or amniotic fluid. DNA can also be obtained from one or more cell or tissue in primary culture, in a propagated cell line, a fixed archival sample, forensic sample or archeological sample. Methods for isolating genomic DNA from a cell, fluid or tissue are well known in the art (see, e.g., Sambrook et al., 2001).

Exemplary cell types from which DNA can be obtained in a method of the invention include, a blood cell such as a B lymphocyte, T lymphocyte, leukocyte, erythrocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which genomic DNA is obtained can be at a particular developmental level including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Other cells include a bone marrow stromal cell (mesenchymal stem cell) or a cell that develops therefrom such as a bone cell (osteocyte), cartilage cells (chondrocyte), fat cell (adipocyte), or other kinds of connective tissue cells such as one found in tendons; neural stem cell or a cell it gives rise to including, for example, a nerve cells (neuron), astrocyte or oligodendrocyte; epithelial stem cell or a cell that arises from an epithelial stem cell such as an absorptive cell, goblet cell, Paneth cell, or enteroendocrine cell; skin stem cell; epidermal stem cell; or follicular stem cell. Generally any type of stem cell can be used including, without limitation, an embryonic stem cell, adult stem cell, totipotent stem cell or pluripotent stem cell.

A cell from which a genomic DNA sample is obtained for use in the invention can be a normal cell or a cell displaying one or more symptom of a particular disease or condition. Thus, a genomic DNA used in a method of the invention can be obtained from a cancer cell, neoplastic cell, apoptotic cell, senescent cell, necrotic cell, an autoimmune cell, a cell comprising a heritable genetic disease or the like.

DNA for use according to the invention may be a standard or reference DNA sample. Such reference samples may comprise a known level of DNA hydroxymethylation. For example, reference DNA samples may be DNA extracted from cells that lack one of more DNA methyltransferase enzyme and are essentially devoid of methylation and hydroxymethylation. In further aspects, a reference DNA sample may be treated with a DNA methyltransferase (e.g., M.SsssI methyltransferase) and an enzyme to convert methylated cytosines into hydroxymethylcytosines (e.g., TET1, TET2 or TET3, see Tahiliani et al., 2009, incorporated herein by reference) and therefore comprise hydroxymethylation at most or essentially all potential methylation sites. For example, a standard DNA may be DNA isolated from the human cell line such as the HCT116 DKO cell line. In certain aspects, methods according to the invention involve the use of two for more standard DNA samples, such as DNA samples comprising essentially no methylation and essentially complete methylation.

### III. METHODS FOR PRODUCING ANTIBODIES

As described above certain aspects of the invention involve antibodies and the use thereof. For example, in some aspects an antibody may be a 5′Glu-hmC-binding antibody that may be used to the presence of 5′Glu-hmC in DNA. Antibodies may be made by any of the methods that as well known to those of skill in the art. The following methods exemplify some of the most common antibody production methods. T he skilled artisan will recognize that the methods provided here may be used to generate antibody that binds 5′Glu-hmC while not binding to 5′mC.

A. Polyclonal Antibodies

Polyclonal antibodies generally are raised in animals by multiple subcutaneous (sc) or intraperitoneal (ip) injections of the antigen. As used herein the term “antigen” refers to any molecule that will be used in the production of antibodies. For example in certain aspects of the invention it is preferred that antibodies recognize 5′Glu-hmC, which for the purposes of antibody production may be coupled to a carrier protein.

It may be useful to conjugate the 5′Glu-hmC antigen to a protein that is immunogenic in the species to be immunized, e.g., keyhole limpet hemocyanin, serum albumin, bovine thyroglobulin, or soybean trypsin inhibitor using a bifunctional or derivatizing agent.

Animals are immunized against the immunogenic conjugates or derivatives by combining 1 mg to 1 μg of conjugate (for rabbits or mice, respectively) with 3 volumes of Freud's complete adjuvant and injecting the solution intradermally at multiple sites. One month later the animals are boosted with 1/5 to 1/10 the original amount of conjugate in Freud's complete adjuvant by subcutaneous injection at multiple sites. 7 to 14 days later the animals are bled and the serum is assayed for specific antibody titer Animals are boosted until the titer plateaus. Preferably, the animal boosted with the same antigen conjugate, but conjugated to a different protein and/or through a different cross-linking reagent. Conjugates also can be made in recombinant cell culture as protein fusions. Also, aggregating agents such as alum are used to enhance the immune response.

B. Monoclonal Antibodies

In certain embodiments of the invention the 5′Glu-hmC-binding antibody is a monoclonal antibody. By using monoclonal a great specificity may be achieved. This may reduce the background in assays of the invention. Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, monoclonal antibodies of the invention may be made using the hybridoma method first described by Kohler et al., 1975, or may be made by recombinant DNA methods (U.S. Pat. No. 4,816,567 to Cabilly et al.).

In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, 1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the target antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., 1980.

After hybridoma cells are identified that produce antibodies of the desired specificity, affinity, and/or activity, the clones may be subcloned by limiting dilution procedures and grown by standard methods, Goding (1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison et al. 1984, or by covalently joining to the immunoglobulin coding sequence all or part of the coding sequence for a non-immunoglobulin polypeptide. In that manner, “chimeric” or “hybrid” antibodies are prepared that have the binding specificity for any particular antigen described herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for the target antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

For diagnostic applications, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; an enzyme, such as alkaline phosphatase, beta-galactosidase or horseradish peroxidase.

Any method known in the art for separately conjugating the antibody to the detectable moiety may be employed, including those methods described by Hunter et al., 1962; David et al., 1974; Pain et al., 1981; and Nygren 1982.

The antibodies of the present invention may be employed in any known assay method, such as competitive binding assays, direct and indirect sandwich assays, and immunoprecipitation assays (Zola, 1987).

Competitive binding assays rely on the ability of a labeled standard (which may be a purified target antigen or an immunologically reactive portion thereof) to compete with the test sample analyte for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected (e.g., AP). In a sandwich assay, the test sample analyte is bound by a first antibody which is immobilized on a solid support, and thereafter a second antibody binds to the analyte, thus forming an insoluble three part complex (see, U.S. Pat. No. 4,376,110). The second antibody may itself be labeled with a detectable moiety (direct sandwich assays) or may be measured using an anti-immunoglobulin antibody that is labeled with a detectable moiety (indirect sandwich assay). In aspects of the invention, such assays may be used to assess AP polypeptide cleavage. One type of sandwich assay is an ELISA assay, in which case the detectable moiety is an enzyme.

### IV. REAGENTS AND KITS

The kits may comprise suitably aliquoted reagents of the present invention, such as a glucosyltransferase (e.g., a β-glucosyltransferase) and one ore more DNA endonucleases (e.g., MspI, TaqI (or TaqαI), or a methylation dependent endonuclease such as BisI, GlaI or McrBC). Additional components that may be included in a kit according to the invention include, but are not limited to, MSEs (e.g., AatII, AccIII, Acil, AfaI, Agel, AhaII, Alw26I, Alw44I, ApaLI, ApyI, Asc1, Asp718I, AvaI, AvaII, Bme216I, BsaAI, BsaHI, BscFI, BsiMI, BsmAI, BsiEI, BsiWI, BsoFI, Bsp105I, Bsp119I, BspDI, BspEI, BspHI, BspKT6I, BspMII, BspRI, BspT104I, BsrFI, BssHII, BstBI, BstEIII, BstUI, BsuFI, BsuRI, CacI, CboI, CbrI, CceI, Cfr10I, ClaI, Csp68KII, Csp45I, CtyI, CviAI, CviSIII, DpnII, EagI, Ec1136II, Eco47I, Eco47III, EcoRII, EcoT22I, EheI, Esp3I, Fnu4III, FseI, FspI, Fsp4III, GsaI, HaeII, HaeIII, HgaI, HhaI, HinPII, HpaII, HpyAIII, Ital, Kas1, Kpn2I, LlaAI, LlaKR2I, MboI, MfII, MluI, MmeII, MroI, MspI, MstII, MthTI, NaeI, NarI, NciAI, NdeII, NgoMIV, NgoPII, NgoS II, NlaIII, NlaIV, NotI, NruI, NspV PmeI, Pm1I, Psp1406I, PvuI, Ra1F40I, RsaI, RspXI, RsrII, SacII, SalI, Sau3AI, SexAI, SfoI, SfuI, SmaI, SnaBI, SolI, SpoI, SspRFI, Sth368I, TaiI, TaqI, TflI, TthHB8I, VpaK11BI, or XhoI), oligonucleotide primers, reference DNA samples (e.g., hydroxymethylated and non-hydroxymethylated reference samples), distilled water, probes, a glucosylation buffer, UDPG, a PCR buffer, dyes, sample vials, polymerase, ligase and instructions for performing methylation assays. In certain further aspects, reagents for DNA isolation, DNA purification and/or DNA clean-up may also be included in a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing reagent containers in close confinement for commercial sale. Such containers may include cardboard containers or injection or blow-molded plastic containers into which the desired vials are retained.

When the components of the kit are provided in one or more liquid solutions, the liquid solution is an aqueous solution, with a sterile aqueous solution being preferred.

However, the components of the kit may be provided as dried powder(s). When reagents and/or components are provided as a dry powder, the powder can be reconstituted by the addition of a suitable solvent. It is envisioned that the solvent may also be provided in another container means.

### V. EXAMPLES

The following examples are included to demonstrate certain embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Methylation Dependent Endonuclease Enzymes Cleave Both 5′mC and 5′hmC

In order to determine if methylation dependent DNA endonucleases could cut at positions comprising both a 5′mC and 5′hmC PCR products were amplified using primers: 5′ (AGA ATT GGT TAA TTG GTT GTA A; SEQ ID NO: 7) and 3′ (ATA TTT GAA TGT ATT TAG AAA AAT AAA; SEQ ID NO: 8). Resulting PCR products have the same primary sequence (SEQ ID NO: 9) and differing only in the modification status of cytosines were digested with the BisI and GlaI endonucleases and analyzed by agarose gel electrophoresis (FIG. 1). Results of the experiment show that, although the BisI cleavage was not complete both enzymes cleaved DNA molecules comprising 5′mC and 5′hmC positions essentially equally.

### Example 2

### Glucosylation of 5′hmC Prevents Cleavage By Methylation Dependent Endonuclease Enzymes

In order to determine if the addition of a larger covalently linked moiety to 5′hmC could inhibit cleavage by a methylation dependent endonucleases, PCR products having the same primary sequence and differing only in the modification status of cytosines were digested with MspI and analyzed by agarose gel electrophoresis (FIG. 2A). Digests were carried out for 3 hours at recommended enzyme reaction conditions either on untreated DNA sample of on samples treated with a β-glucosyltransferase from T4 bacteriophage. The results show that addition of the glucose to 5′hmC effectively inhibited MspI cleavage. Furthermore, results from FIG. 2A demonstrate that glucosylation was specific to 5′hmC and was very efficient, in that essentially all of the DNA was protected from cleavage.

### Example 3

### Hemi-Glu-5′hmC Prevents Cleavage By Methylation Dependent Endonuclease Enzymes

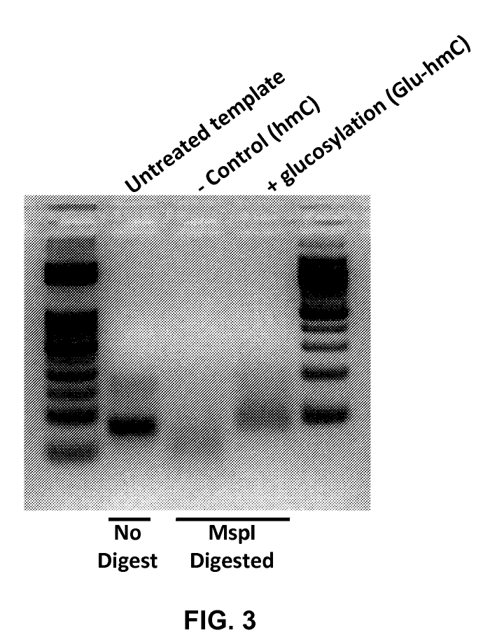

In order to determine whether glucosylation of 5′hmC both strands of DNA was required to inhibit cleavage, a DNA template with hemi-Glu-5′hmC (TAAAAGCTAACCGCATCTTTACCGACAAGGCATCCGGCAGTTCAACAGATCGGG AAGGGCTGGATTTGCTGAGGATGAAGGTGGA; SEQ ID NO: 10, underlined “C” was modified to Glu-5′hmC) was digested with MspI and analyzed by agarose gel electrophoresis. The results shown in FIG. 3 demonstrate that hemi-Glu-5′hmC effectively blocks MspI digestion.

### Example 4

### Glu-5′hmC Prevents Cleavage By TaqαI Digestion

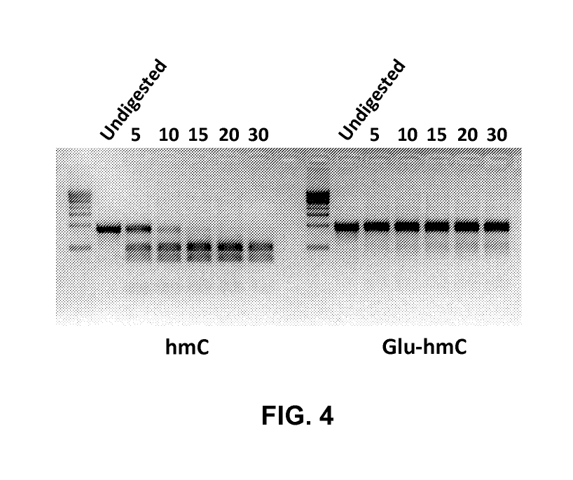

In order to determine the ability of Glu-5′hmC to inhibit digestion with additional endonuclease enzymes DNA templates comprising Glu-5′hmC or 5′hmC were digest with TaqαI (recombinant TaqI). Results shown in FIG. 4 demonstrate that TakαI digestion was inhibited only by Glu-5′hmC.

### Example 5

### Glu-5′hmC Prevents Cleavage By MspI After in vitro Conversion of Unmodified Cytosines to Glu-5′hmC

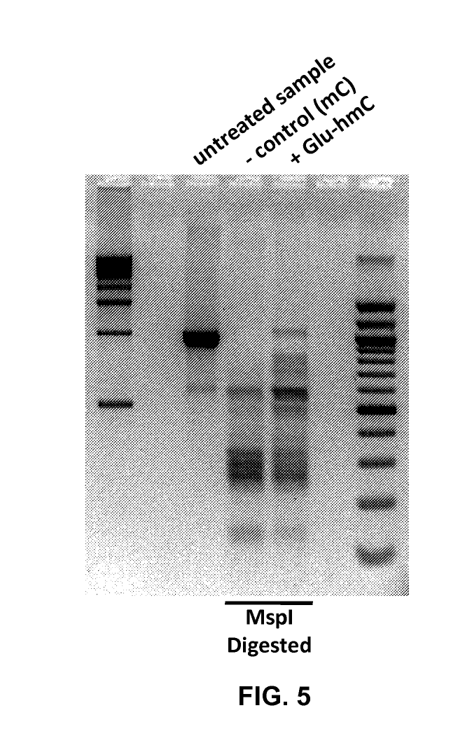

A DNA template containing all unmodified cytosines was in vitro methylated at CpG sites with M.SssI. CpG methylated template was treated in vitro with Tet1 to create 5′-hydroxymethylcytosine on the premethylated (mCpG) sites. Then mC and mC+Tet1 (hmC) samples were glucosylated with β-glucosyltransferase and subsequently digested with MspI. As shown in FIG. 5 only the DNA treated with Tet1 contains 5′hmC which could accept a glucose moiety.

### Example 6

### DNA Comprising Glu-5′hmC is a Suitable Substrate for PCR

To determine if DNA comprising Glu-5′hmC could be amplified by PCR sample template was amplified from pUC18 (using primers pUC 5′ (ttttaaattaaaaatgaagttttaaat; SEQ ID NO: 4) and pUC 3′ (aataatattgaaaaaggaagagtatgagtatt; SEQ ID NO: 5)). The resulting PCR product has the sequence of SEQ ID NO: 6. A portion of the PCR product was left untreated (C) and a portion was in vitro methylated with M.SssI to create sample “mC”. Part of sample “mC” was treated in vitro with Tet1 to create hydroxymethylcytosine on pre-methylated C's. Then sample “mC” along with the Tet1 treated sample (containing hmC) were in vitro glucosylated with β-glucosyltransferase. Thus, the sample labeled “GluhmC” contains glucosyl-5′-hydroxymethylcytsoine because only the +Tet1 sample will accept glucosyl groups.

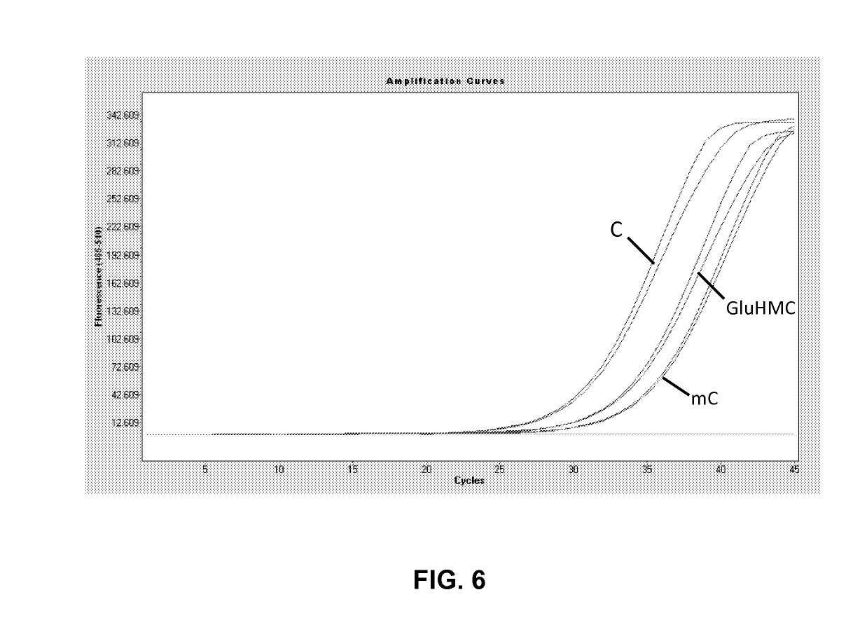

qRT-PCR was performed in duplicates with 4 pg of “C,” “mC” and “GluhmC” input DNA for each template. The results indicate shown in FIG. 6 and Table 1 show that DNA containing glucosyl-5-hydroxymethylcytosine is efficiently amplified via PCR similar to DNA comprising methylated cytosine positions.

TABLE 1
Quantification of the qRT-PCR amplification

| Sample | Cp | Avg. Cp |
|---|---|---|
| C | 31.57 | 31.62 |
| C | 31.66 | |
| mC | 35.97 | 35.96 |
| mC | 35.94 | |
| GluhmC | 34.5 | 34.55 |
| GluhmC | 34.6 | |
| No DNA | — | — |
| No DNA | — | |

### Example 7

### Glu-5′hmC Can Be Used to Quantify Hydroxymethylation in a DNA Sequence

To test whether glucosylation of 5′-hydroxymethycytosine can be used to gauge for locus specific quantification of 5′-hydroxymethylcytosine, DNA “-Control DNA” from Example 5 (FIG. 5), containing methylated cytosines in CpG context and “+Tet” DNA samples, containing glucosyl-5′-hydroxymethylcytosine, were first digested with MspI. Then the MspI digested DNA were analyzed for amplification efficiency by qRT-PCR. Both sample input were at 500 pg per reaction.

Quantification of the results is shown in Table 2. The study demonstrates that glucosylated-5′-hydroxymethylcytosine DNA amplified ˜4.2 cycles before 5′-methylcytosine containing DNA, indicating a greater than 16-fold enrichment of glucosylated-5′-hydroxymethylcytosine DNA after MspI digestion. These results show that glucosylation of DNA coupled to DNA endonuclease digestion and quantitated by qRT-PCR offers a reliable method for locus specific quantification of 5′ -hydroxymethylcytosine. Results also clearly demonstrate that DNA comprising Glucosylated-5′-hydroxymethylcytosine can be amplified by PCR and that PCR can detect hydroxymethylated DNA positions relative to methylated positions in a sample after enzyme digestion.

TABLE 2
Quantification of the qRT-PCR amplification

| Sample | Cp | Avg. Cp |
|---|---|---|
| Tet1 (GluHMC) | 28.73 | 28.66 |
| Tet1 (GluHMC) | 28.59 | |
| -Cont (5mC) | 32.96 | 32.86 |
| -Cont (5mC) | 32.76 | |
| No DNA Input | — | — |

### Example 8

### Cloning and Glucosylation with β-Glucosyltransferase

The coding region for T4 β-glucosyltransferase was amplified using oligonucleotide primers 5′ (atgaaaattgctataattaatatgg; SEQ ID NO: 1) and 3′ (ttataaatcaatagcttttttgaac; SEQ ID NO: 2) resulting in a coding region having the sequence of SEQ ID NO: 3. The coding sequence was subcloned into an expression vector; over expressed and purified using standard techniques (see, e.g., Tomaschewski et al., 1985).

In vitro glucosylation reactions were carried out in the presence of uridine diphosphate glucose (UDPG) in an appropriate buffer. For example, a 1× reaction buffer may comprise 50 mM Tris (pH 7.5), 25 mM MgCl₂ 1 mM DTT and 100 μM UDPG or may comprise 50 mM Potassium Phosphate buffer (pH 7.6), 25 mM MgCl₂, 1 mM DTT and 100 μM UDPG. An example reaction mix is provided below.

| DNA [100 ng/μl] | 10 μl (1 μg) | |
|---|---|---|
| 10xBgt Rxn Bfr | 5 | μl |
| [10 mM]100xUDPG | 0.5 | μl |
| β-glucosyltransferase | 1 | μl |
| ddH₂O | 33.5 | μl |
| Total Vol | 50 | μl |

DNA was found to be effectively glucosylated after incubation for 1 hour at 30° C.

Another example of Glucosylation reaction is provided below:

| | |
|---|---|
| DNA [10-100 ng/μl] | 10 μl |
| 10X 5hmC GT Reaction Buffer | 5 μl |
| 10X UDPG [1 mM] | 5 μl |
| 5hmC GT Enzyme (2 units/μl) | 2 μl |
| ddH2O | 28 μl |
| Total | 50 μl |

A standard reaction setup shown above would incubation at 30° C. for ≧2 hours.

To ensure glucosylation reaction is carried to completion excess enzyme unit:DNA ratio may be used. For example, if glucosylating 1 μg of DNA use 4 units of 5′hmC Glucosyltransferase. Likewise the reaction may be extended for an incubation at 30° C. for ≧2 hours.

Reactions such as those above may be used for global quantification of 5′hmC with use of Uridine Diphosphate Glucose [Glucose-¹⁴C(U)] PerkinElmer (Szwagierczak et al., 2010).

### Example 9

### Example Kit and Protocol for Detection of DNA Hydroxymethylation

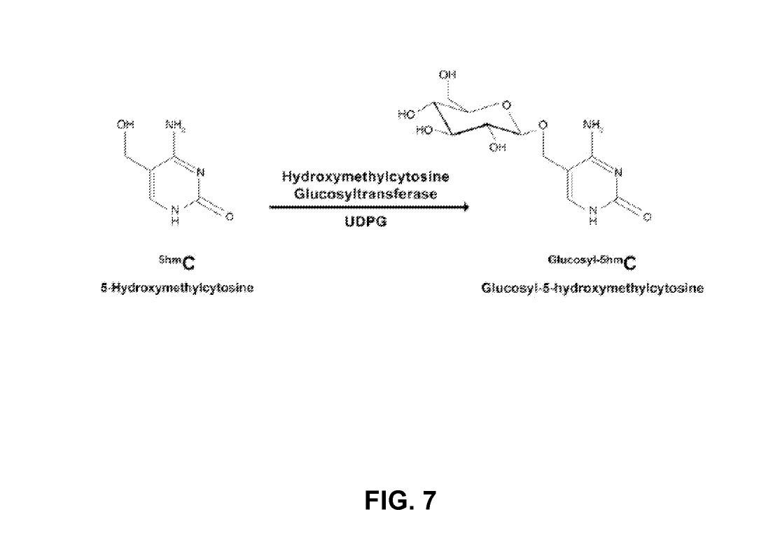

A kit according to the invention uses a robust and highly specific 5-hmC Glucosyltransferase enzyme. 5-hydroxymethylcytosine in DNA is specifically tagged with a glucose moiety yielding a modified base, glucosyl-5-hydroxymethylcytosine (FIG. 7).

After glucosylation of 5-hydroxymethylcytosine, digestion of DNA with “5-hydroxymethylcytosine sensitive” restriction endonucleases, or GSRE's (see, e.g., Table 3), allows for effective differentiation of 5-methylcytosine from 5-hydroxymethylcytosine. Identification of 5-hydroxymethylcytosine in a sequence specific context can then be deduced from the restriction endonuclease recognition sequence (Table 3).

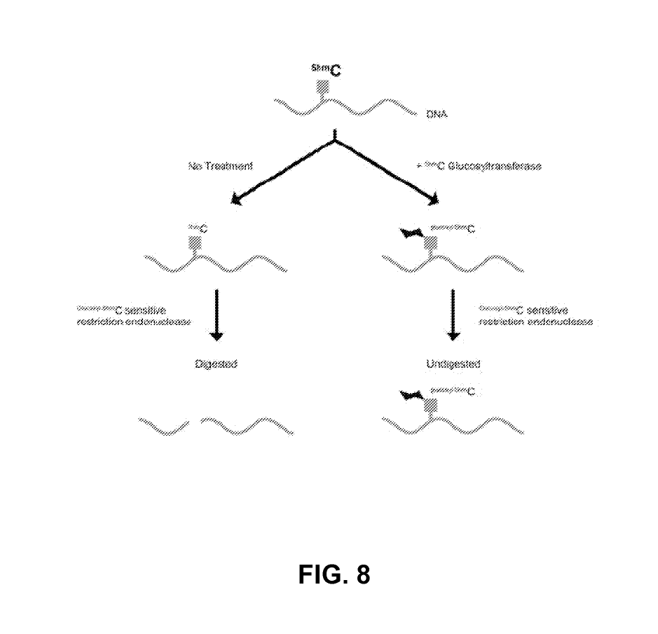

Included in a kit is a GSRE such as GlaI (for others see Table 3). GlaI is also a methylation dependent restriction endonuclease that can digest DNA only when 5′-methylcytosine or 5′-hydroxymethylcytosine lies within its recognition sequence. However, when 5′-hydroxymethylcytosine is glucosylated (glucosyl-5-hydroxymethylcytosine), GlaI is no longer able to digest (FIG. 2B). A general protocol is shown in FIG. 8.

TABLE 3
Example GSREs.

| GSRE | Recognition Sequence |
|---|---|
| GlaI | GCGC |
| | ACGC |
| | ACGT |
| MspI | CCGG |
| Taq^{α}I * | TCGA |

* TaqαI displays incomplete sensitivity to Glucosyl-5′hmC. Enzyme and incubation time titration may be needed for optimal results.

After processing of DNA with a 5′-hmC detection kit, detection of 5′hmC sites can be achieved by a variety of techniques such as: qPCR, ultra-deep sequencing, southern blot and microarray.

Eluted DNA containing 5-hydroxymethylcytosine residues will be fully glycosylated (glucosyl-5-hydroxymethylcytosine). Amplification of glucosyl-5-hydroxymethylcytosine containing DNA displays lower amplification efficiencies with some Taq DNA polymerases. However, PCR mixtures can be optimized specifically for efficient amplification of DNA templates containing glucosyl-5-hydroxymethylcytosine residues.

The following two protocols describe a streamlined method for 5′hmC detection. DNA sample preparation entailing glucosylation of 5′hmC within DNA, methylation of DNA (used in the GlaI method), and subsequent digestion of DNA with GSRE's is carried out in a one tube format.

For use with GlaI, a DNA methlytransferase cocktail must be used. GlaI is a methylation dependent restriction endonuclease, and can only digest DNA effectively when DNA is fully methylated. Conversely, for use with MspI, no DNA methlytransferase cocktail is required. Methylation patterns induced by the DNA methlytransferase cocktail will inhibit cutting of some MspI sites.

A DNA methyltransferase cocktail can be formulated as a mixture of CpG (M.SssI) and GpC (M.CviPI) DNA methyltransferases.

GlaI Protocol:

Note: GlaI is a methylation dependent endonuclease, therefore use of DNA Methyltransferase Cocktail is necessary for complete GlaI digestion.

1. Standard reaction setup shown below. Incubate at 30° C. for ˜2 hours.

| | | |
|---|---|---|
| DNA [10-100 ng/μl] | 10 | μl |
| 10X 5-hmC GT Reaction Buffer | 5 | μl |
| 10X UDPG [1 mM] | 5 | μl |
| 5-hmC GT Enzyme (2 units/μl) | 2 | μl |
| DNA Methyltransferase Cocktail (2 units/μl) | 1.5 | μl |
| 20X SAM [12 mM] | 2.5 | μl |
| ddH2O | 24 | μl |
| Total | 50 | μl |

2. After ˜2 hour incubation in Step 1, add 1 μl (4 units) GlaI Restriction Enzyme directly to reaction. Incubate at 30° C. for 6-16 hours

3. Add a 5:1 ratio DNA Binding Buffer to the reaction (e.g., 250 μl DNA Binding Buffer to a 50 μl reaction volume)

4. Proceed directly to Step 2 in the “Protocol” section of DNA Clean & Concentrator™ (or other DNA purification system).

MspI Protocol:

Note: Do not add DNA Methyltransferase Cocktail to reaction for MspI. DNA methylation profile induced by DNA Methyltransferase Cocktail may interfere with MspI digest.

1. Standard reaction setup shown below. Incubate at 30° C. for ˜2 hours.

| | |
|---|---|
| DNA [10-100 ng/μl] | 10 μl |
| 10X 5-hmC GT Reaction Buffer | 5 μl |
| 10X UDPG [1 mM] | 5 μl |
| 5-hmC GT Enzyme (2 units/μl) | 2 μl |
| ddH2O | 28 μl |
| Total | 50 μl |

2. After ˜2 hour incubation in Step 1, add 10 units of MspI restriction enzyme (not included) directly to reaction. Incubate at 37° C. for ˜2 hours.

3. Add a 5:1 ratio DNA Binding Buffer to the reaction (e.g., 250 μl DNA Binding Buffer to a 50 μl reaction volume)

4. Proceed directly to Step 2 in the “Protocol” section of DNA Clean & Concentrator™ (or other DNA purification system).

### REFERENCES

Each of the foregoing documents is hereby incorporated by reference in its entirety:
U.S. Pat. Nos. 4,376,110; 4,816,567; 5,436,134 and 5,658,751.
David et al., Biochemistry, 13:1014, 1974.
Goding, In: Monoclonal Antibodies: Principles and Practice, 60-61, 71-74, 1986.
Huang et al., PLoS ONE, 5(1):e8888, 2010.
Hunter et al., Nature, 144:945, 1962.
Jones et al., Nat. Genet., 21(2):163-7, 1999.
Kohler et al., Nature, 256:495-497, 1975.
Morrison et al., Proc. Natl. Acad. Sci. U.S.A., 81:6851, 1984.
Munson et al., Anal. Biochem., 107:220, 1980.
Nestor et al., BioTechniques, 48(4):317-319, 2010.
Nygren, J. Histochem. Cytochem., 30(5):407-412, 1982.
Oakes et al., Epigenetics, 1(3):146-152, 2009.
Pain et al., J. Immunol. Meth., 40:219, 1981.
PCT Publn. WO/2010/114821
Sambrook et al., In: Molecular Cloning-A Laboratory Manual, 1989.
Szwagierczak et al, Nucleic Acids Res., 1-5, 2010.
Tahiliani et al., Science, 324:930-935, 2009.
Tomaschewski et al., Nuc. Acids Res., 13(21):7551-7568, 1985.
Zola, In: Monoclonal Antibodies. A Manual of Techniques, 147-158, 1987.

1. A method for detecting DNA hydroxymethylation in a DNA sample comprising:
(i) contacting a DNA sample comprising a glycosylated hydroxymethylcytosine with a DNA endonuclease to cleave the DNA; and
(ii) detecting at least a first DNA sequence from a sample not cleaved by the DNA endonuclease to determine the presence of hydroxymethylation in the DNA sequence.

2. The method of claim 1, wherein contacting the DNA sample with a DNA endonuclease comprises contacting the DNA with two or more DNA endonucleases.

3. The method of claim 1 wherein the DNA endonuclease is a methylation dependent DNA endonuclease.

4. The method of claim 3, wherein contacting the DNA sample with a methylation dependent DNA endonuclease comprises contacting the DNA with two or more methylation dependent DNA endonucleases.

5. The method of claim 1, wherein detecting DNA sequences not cleaved by the DNA endonuclease comprises DNA sequencing or hybridization.

6. The method of claim 1, further comprising ligating the cleaved DNA to an oligonucleotide tag before said detecting of step (ii), wherein the oligonucleotide tag comprises a sequence for PCR primer binding.

7. The method of claim 6, wherein the oligonucleotide tag comprises a label.

8. The method of claim 7, wherein the label is a fluorescent, a colorimetric, an enzymatic, an antigen or a radioactive label.

9. The method of claim 6, wherein detecting DNA sequences not cleaved by the DNA endonuclease comprises sequencing the DNA using a primer that hybridizes to the oligonucleotide tag.

10. The method of claim 1, comprising determining the presence of DNA hydroxymethylation at a plurality of potential hydroxymethylation sites.

11. The method of claim 10, comprising determining the presence of DNA hydroxymethylation at least 5, 10, 15, 20, 50, 100, 500 or 1,000 potential hydroxymethylation sites.

12. The method of claim 10, wherein determining the presence of DNA hydroxymethylation at a potential methylation site comprises identifying a sequence corresponding detected DNA sequence on a genomic map.

13. The method of claim 1, further comprising detecting DNA hydroxymethylation in two or more DNA samples to determine differential DNA hydroxymethylation between two or more samples.

14. The method of claim 13, wherein said two or more samples comprise: samples from tissue culture cells grown under different conditions; samples from cells at different stages of differentiation; samples from healthy and diseases tissue; samples two or more different organisms or individuals; or samples from cells treated with a drug and placebo.

15. The method of claim 1, wherein the DNA sample comprises mammalian genomic DNA.

16. The method of claim 15, wherein the mammalian genomic DNA is human genomic DNA.

17. The method of claim 16, wherein the human genomic DNA is from a human subject.

18. The method of claim 15, wherein the human genomic DNA is from a cell line or tissue bank.

19. The method of claim 1, wherein the DNA sample is from cultured cells, a tissue biopsy blood, urine, saliva or skin.

20. The method of claim 19, wherein the cultured cells are neuronal cells or stem cells.

21. The method of claim 1, wherein the DNA endonuclease is MspI, BisI, GlaI, TaqαI or McrBC.

22. The method of claim 3, wherein the methylation dependent DNA endonuclease is BisI, GlaI or McrBC.

23. The method of claim 1, further comprising contacting the DNA sample with a methylation sensitive DNA endonuclease (MSE) before step (ii).

24. The method of claim 23, further comprising contacting the DNA sample with a methylation sensitive DNA endonuclease (MSE) before step (i) and detecting DNA sequences not cleaved by the methylation sensitive DNA endonuclease to determine the presence of DNA methylation.

25. The method of claim 1, further comprising treating a DNA sample to glycosylate hydroxymethylcytosine positions before step (i).

26. The method of claim 25, wherein treating the DNA sample to glycosylate hydroxymethylcytosine positions comprises contacting the DNA sample with a glucosyltransferase.

27. The method of claim 26, wherein the glucosyltransferase is recombinant.

28. The method of claim 26, wherein the glucosyltransferase is from a T-even bacteriophage.

29. The method of claim 26, wherein the glucosyltransferase is a β-glucosyltransferase.

30. The method of claim 1, further comprising contacting the DNA sample with a DNA methyltransferase prior to step (i).

31. The method of claim 30, wherein the DNA methyltransferase is M.SssI or M.CviPI.

32. A method for detecting DNA methylation and hydroxymethylation in a DNA sample comprising:
(i) contacting a DNA sample with a methylation sensitive DNA endonuclease (MSE) to cleave the DNA;
(ii) contacting the cleaved DNA sample comprising a glycosylated hydroxymethylcytosine with a methylation dependent DNA endonuclease to cleave the DNA; and
(iii) detecting DNA sequences not cleaved by the methylation dependent DNA endonuclease to determine the presence of hydroxymethylation.

33-47. (canceled)

48. A substantially purified mammalian DNA sample comprising at least one glucosylated hydroxymethylcytosine.


SEQUENCE LISTING (10 sequences)

SEQ ID NO: 1 - DNA, 25 bp, Artificial Sequence
  Synthetic primer
atgaaaattgctataattaatatgg

SEQ ID NO: 2 - DNA, 25 bp, Artificial Sequence
  Synthetic primer
ttataaatcaatagcttttttgaac

SEQ ID NO: 3 - DNA, 1056 bp, Bacteriophage T4
atgaaaattgctataattaatatgggtaataatgttattaattttaaaactgttccatcttctgaaactatttatctttttaaagttatttctgaaatgggtcttaatgtcgacattatttctcttaaaaatggtgtttacactaaatcttttgatgaagtagatgttaatgattatgaccgtttgatagttgttaattcttctattaacttttttggcggtaaacctaatttagcaattttatctgcgcaaaaatttatggcaaaatacaaaagtaaaatttattatttatttacagatatacgtttgccgttttcgcagtcttggccaaatgttaaaaatagaccatgggcatatttgtacactgaagaagagctattaattaaatcaccaattaaagtgatttcccaaggtataaatttagacattgctaaggctgcgcataagaaagttgataatgttattgaatttgaatattttcctattgaacaatataaaattcatatgaacgattttcaattatctaagcctaccaagaaaactttggatgttatttatggcggttcatttcggtccggtcaacgcgaatccaagatggtagaattcttatttgacaccggtttaaatattgagttttttggcaatgcacgagaaaaacagtttaaaaatcctaaatatccttggaccaaagctccggtgttcactggaaaaattcctatgaacatggtatctgaaaagaatagtcaagctattgctgcattaattattggtgacaagaattataatgacaactttattaccttacgcgtctgggaaacaatggcatctgatgcagtgatgctaattgacgaagaatttgataccaaacatcgaattattaatgatgctcgtttttatgtaaataatcgtgctgaactcattgatagagtcaatgagttaaaacacagtgatgttttgcgtaaagagatgctttctattcaacatgatattttaaataaaacccgtgcaaagaaagccgaatggcaagatgcgttcaaaaaagctattgatttataa

SEQ ID NO: 4 - DNA, 27 bp, Artificial Sequence
  Synthetic primer
ttttaaattaaaaatgaagttttaaat

SEQ ID NO: 5 - DNA, 32 bp, Artificial Sequence
  Synthetic primer
aataatattgaaaaaggaagagtatgagtatt

SEQ ID NO: 6 - DNA, 948 bp, Artificial Sequence
  PCR amplification product
ttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattatt

SEQ ID NO: 7 - DNA, 22 bp, Artificial Sequence
  Synthetic primer
agaattggttaattggttgtaa

SEQ ID NO: 8 - DNA, 27 bp, Artificial Sequence
  Synthetic primer
atatttgaatgtatttagaaaaataaa

SEQ ID NO: 9 - DNA, 897 bp, Artificial Sequence
  PCR amplificatio product
agaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatat

SEQ ID NO: 10 - DNA, 85 bp, Artificial Sequence
  DNA template sequence
taaaagctaaccgcatctttaccgacaaggcatccggcagttcaacagatcgggaagggctggatttgctgaggatgaaggtgga